(19) United States
(12) Patent Application Publication
(10) Pub. No.: US 2007/0191839 A1
(43) Pub. Date: Aug. 16, 2007

(54) Non-locking multi-axial joints in a vertebral implant and methods of use

(75) Inventors: Jeff R. Justis, Germantown, TN (US); Fred J. Molz IV, Birmingham, AL (US)

Correspondence Address: COATS & BENNETT, PLLC, 1400 Crescent Green, Suite 300, Cary, NC, 27518, US

(73) Assignee: SDGI Holdings, Inc.

(21) Appl. No.: 11/341,239

(22) Filed: Jan. 27, 2006

(51) Int. Cl. A61F 2/30

(52) U.S. Cl. 606/61

(57) ABSTRACT

A connector pivotally connects an anchor to a longitudinal member in a spinal implant. The connector body may include an oppositely disposed channel and cavity that are aligned on a common axis of the body, but isolated from each other. The channel receives the longitudinal member and the cavity receives the anchor. The anchor may include a shaft and an enlarged head that fits within the cavity. The cavity may include a narrow opening that is sized to retain the head within the cavity. The head may pivot within a wear member. The anchor may freely pivot within the cavity when a fastener mates with the receiver to maintain the longitudinal member within the channel.

### BACKGROUND

Longitudinal members, such as spinal rods, are often used in the surgical treatment of spinal disorders such as degenerative disc disease, disc herniations, scoliosis or other curvature abnormalities, and fractures. Different types of surgical treatments are used. In some cases, spinal fusion is indicated to inhibit relative motion between vertebral bodies. In other cases, dynamic implants are used to preserve motion between vertebral bodies. For either type of surgical treatment, longitudinal members may be attached to the exterior of two or more vertebrae, whether it is at a posterior, anterior, or lateral side of the vertebrae. In other embodiments, longitudinal members are attached to the vertebrae without the use of dynamic implants or spinal fusion.

Longitudinal members may provide a stable, rigid column that encourages bones to fuse after spinal-fusion surgery. Further, the longitudinal members may redirect stresses over a wider area away from a damaged or defective region. Also, rigid longitudinal members may restore the spine to its proper alignment. In some cases, a flexible longitudinal member may be appropriate. Flexible longitudinal members may provide other advantages, such as increasing loading on interbody constructs, decreasing stress transfer to adjacent vertebral elements while bone-graft healing takes place, and generally balancing strength with flexibility.

Conventionally, longitudinal members are secured to vertebral members using rigid clamping devices. These clamping devices may be multi-axial in the sense that they are adjustable prior to securing. However, once secured, the clamping devices are locked in place. A surgeon may wish to implant a flexible rod system and have more freedom to control pivot points or the nature of the pivoting motion. At present, a surgeon might only have a choice between rigid and flexible longitudinal members, which may not necessarily provide the desired degree of flexibility.

### SUMMARY

Illustrative embodiments disclosed herein are directed to a connector that pivotally connects a vertebral anchor to a longitudinal member. The connector body may be directly or indirectly attached to the anchor. The connector body may include a channel and a cavity that are aligned along a common axis. The channel is generally sized to receive the longitudinal member. The connector may have an associated fastener that mates with the channel to maintain the longitudinal member in the channel. The cavity may be positioned on an opposite side of the body from the channel while being aligned with the channel. Further, the cavity may include a narrow opening that extends into an enlarged receiving area. The receiving area may be isolated from the channel. In one embodiment, an intermediate section defines a boundary between the receiving area and the channel. The receiving area may be sized to accommodate an enlarged head of the anchor. The narrow opening may be sized to retain the head within the receiving area. The receiving area may be further sized to allow the anchor to freely pivot about the common axis, even when the fastener mates with the receiver. The connector may also include a wear member positioned within the cavity. The wear member may form its own receiving area that is isolated from the channel and sized to accommodate the head of the anchor.

### BRIEF DESCRIPTION OF THE DRAWINGS

FIGS. 1A and 1B are perspective views of an assembly according to one or more embodiments comprising a longitudinal member attached to the spine;

FIGS. 2A and 2B are perspective views of a pivoting head coupled to an anchor member according to one embodiment;

FIG. 3 is a side section view of a pivoting head coupled to an anchor member and securing a longitudinal member according to one embodiment;

FIG. 4 is a perspective view of an anchor member for use with a pivoting head according to one embodiment;

FIG. 5 is a perspective view of a wear member for use with a pivoting head according to one embodiment;

FIG. 6 is a side view, including a partial section view, of an assembled anchor member and wear member for use with a pivoting head according to one embodiment;

FIG. 7 is a side section view of a pivoting head with an anchor member and wear member inserted therein according to one embodiment;

FIG. 8 is a side section view of an assembled pivoting head with an anchor member and wear member constrained therein according to one embodiment;

FIGS. 9A and 9B are top section views of a pivoting head with an anchor member and wear member inserted therein according to different embodiments;

FIG. 10 is a side section view of an assembled pivoting head with an anchor member and wear member constrained therein according to one embodiment;

FIG. 11 is a perspective view of a wear member for use with a pivoting head according to one embodiment;

FIG. 12 is a side section view of an unassembled anchor member and wear member for use with a pivoting head according to one embodiment;

FIGS. 13A and 13B are side section views of an assembled anchor member and wear member for use with a pivoting head according to one embodiment;

FIGS. 14A and 14B are side section views showing a technique for assembling a pivoting head with an anchor member and wear member constrained therein according to one embodiment;

FIG. 15 is a side section view of an assembled pivoting head with an anchor member and wear member constrained therein according to one embodiment; and

FIG. 16 is a side section view of an assembled pivoting head with an anchor member and wear member constrained therein according to one embodiment.

### DETAILED DESCRIPTION

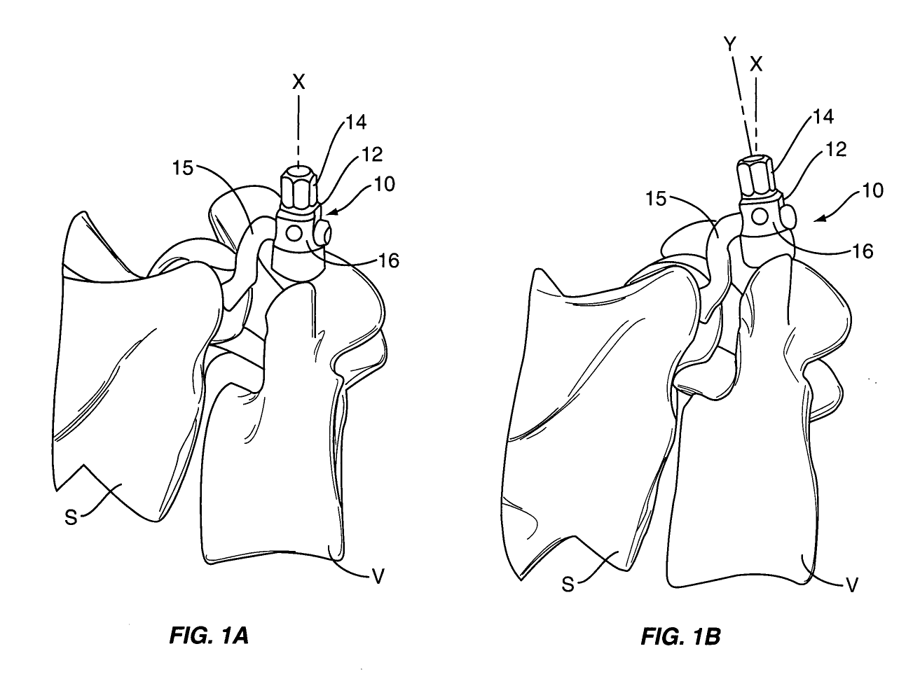

The various embodiments disclosed herein are directed to non-locking, multi-axial clamping mechanisms for securing longitudinal members. Various types of longitudinal members are contemplated, including spinal rods that may be secured between multiple vertebral bodies. FIGS. 1A and 1B show another type of longitudinal member 15 that is secured between the sacrum S and a vertebral member V (e.g., L5). In one embodiment, the longitudinal member 15 is a flexible member, such as a resin or polymer compound. Some flexible non-metallic longitudinal members 15 are constructed from materials such as PEEK and UHMWPE. Other types of flexible longitudinal members 15 may comprise braided metallic structures. In one embodiment, the longitudinal member 15 is rigid or semi-rigid and may be constructed from metals, including for example stainless steels, cobalt-chrome, titanium, and shape memory alloys. Further, the longitudinal member 15 may be straight, curved, or comprise one or more curved portions along its length.

In FIGS. 1A and 1B, the longitudinal member 15 is secured to the vertebral member V with one embodiment of a non-locking, pivoting head 10 in accordance with the teachings provided herein. In the embodiment shown, the longitudinal member 15 is secured to a saddle 16 within the pivoting head 10 with a securing member 12. The securing member 12 shown in FIGS. 1A and 1B features a snap-off driving member 14. The driving member 14 is integrally formed with the securing member 12 and allows a surgeon to drive the securing member 12 into contact with the longitudinal member 15 to achieve a certain installation torque. Above that torque, the driving member 14 will snap off, separating from the securing member 12. In this manner, the securing member 12 may provide the desired clamping force to secure the longitudinal member 15.

FIG. 1A shows a first orientation for the pivoting head 10 identified by the centerline labeled X. By contrast, FIG. 1B shows a second position representing a different spatial relationship between the sacrum S and the vertebra V. As compared to FIG. 1A, the vertebra V in FIG. 1B exhibits some amount of angular and torsional displacement relative to the sacrum S. Consequently, the pivoting head 10 is illustrated in a second orientation identified by the centerline labeled Y. The pivoting head 10 may provide some or all of this rotation. The illustrations provided in FIGS. 1A and 1B show the pivoting head 10 as part of a spinal implant that is coupled between a vertebral body V and a sacrum S. It should be understood that the pivoting head 10 may be used in constructs that are coupled to vertebral bodies V alone. Further, a vertebral implant may be construed to mean implants that are coupled to any or all portions of a spine, including the sacrum, vertebral bodies, and the skull.

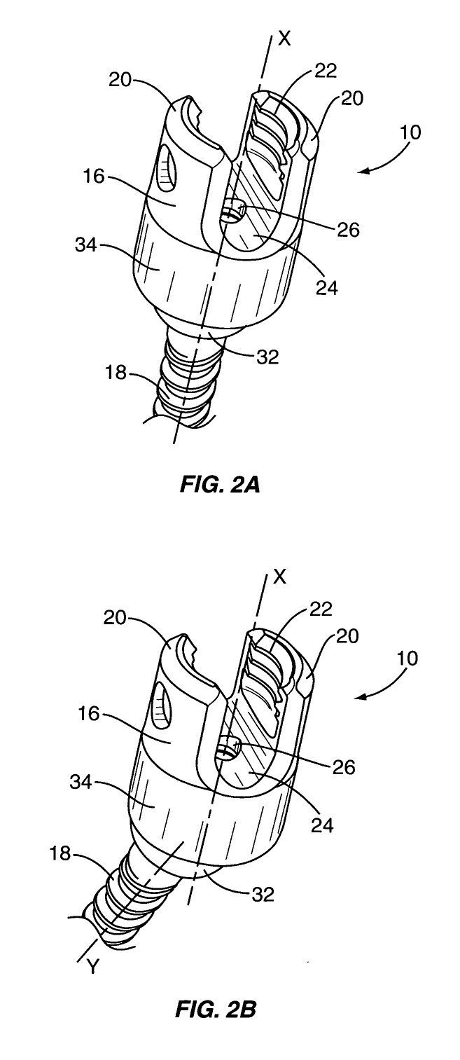

FIGS. 2A and 2B illustrate perspective views of the illustrative embodiment of the pivoting head 10 coupled to an anchor member 18. A head 32 of the anchor member 18 is pivotally coupled to a base portion 34 of the pivoting head 10. In one embodiment, the anchor member 18 comprises threads for insertion into a vertebral member V as shown in FIG. 1. In one embodiment, the anchor member 18 is a pedicle screw. The exemplary saddle 16 is comprised of opposed upright portions forming a U-shaped channel within which a longitudinal member 15 is placed. A seating surface 24 forms the bottom of the U-shaped channel. In one embodiment, the seating surface 24 is curved to substantially match the radius of a longitudinal member 15 that is positioned within the saddle 16. An aperture 26 within the seating surface provides access to a driving feature used to insert the anchor member 18 into a vertebral member V.

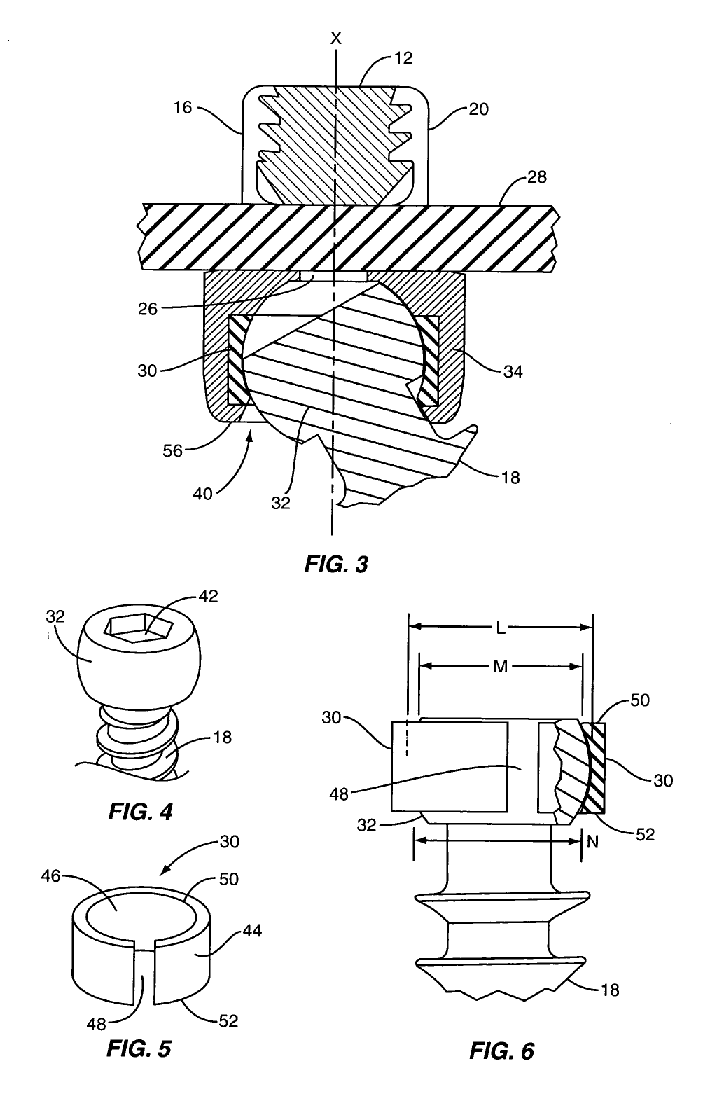

In FIG. 2A, the pivoting head 10 is shown substantially aligned with the anchor member 18 along the centerline labeled X. In FIG. 2B, the anchor member 18 is shown pivoted relative to the pivoting head 10. That is, the pivoting head 10 is shown still aligned with the centerline labeled X while the anchor member 18 is shown aligned with the centerline labeled Y. The pivoted displacement of the pivoting head 10 relative to the anchor member 18 achieved in FIG. 2B is provided by an articulation mechanism that is more clearly visible in the section view provided in FIG. 3.

FIG. 3 shows a section view of the pivoting head 10 holding a different type of longitudinal member 28. In this embodiment, the longitudinal member 28 is a spinal rod. The spinal rod 28 is secured within the saddle 16 with a securing member 12. In the embodiment shown, the securing member 12 is an externally threaded set screw, though other types of securing members such as externally threaded caps and nuts may be used. In the embodiment shown, an articulation mechanism 40 is disposed below the saddle 16 and generally aligned with the central axis X. The articulation mechanism 40 comprises an enlarged head 32 of the anchor member 18 that is pivotally coupled to a wear member 30 within the base portion 34 of the pivoting head 10. Since the enlarged head 32 is configured to pivot within the wear member 30, the wear member 30 and the outer surface of the enlarged head 32 may be constructed of a wear resistant material. Some suitable examples may include hardened metals, titanium carbide, cobalt chrome, polymers, and ceramics.

In other embodiments, a wear resistant layer may be coated onto the enlarged head 32 and the wear member 30. In one embodiment, the wear member 30 may be integrally formed into or form a part of the base portion 34. In one embodiment, the wear member 30 may be bonded to the base portion 34 using a biocompatible adhesive such as PMMA or other known adhesives. In these alternative embodiments, the part of the base portion 34 in contact with the enlarged head 32 may be coated with a wear resistant layer. Coating processes that include, for example, vapor deposition, dip coating, diffusion bonding, and electron beam welding may be used to coat the above indicated materials onto a similar or dissimilar substrate. Diffusion bonding is a solid-state joining process capable of joining a wide range of metal and ceramic combinations. The process may be applied over a variety of durations, applied pressure, bonding temperature, and method of heat application. The bonding is typically formed in the solid phase and may be carried out in vacuum or a protective atmosphere, with heat being applied by radiant, induction, direct or indirect resistance heating. Electron beam welding is a fusion welding process in which a beam of high-velocity electrons is applied to the materials being joined. The workpieces melt as the kinetic energy of the electrons is transformed into heat upon impact. Pressure is not necessarily applied, though the welding is often done in a vacuum to prevent the dispersion of the electron beam.

The articulation mechanism 40 is spatially and functionally isolated from the clamping forces that are applied between the securing member 12, the rod 28, and the seating surface 24 (see FIGS. 2A, 2B). That is, since the compression forces applied by the securing member 12 are not transmitted to the articulation mechanism 40, the anchor member 18 freely rotates about the central axis X. In one embodiment, there is no interference between the enlarged head 32 and the wear member 30. This type of fit may minimize the sliding friction that impedes the motion of the anchor member 18 relative to the wear member 30.

FIG. 4 shows a perspective view of the enlarged head 32 of the exemplary anchor member 18. In this illustrated embodiment, the enlarged head 32 is substantially spherical to allow multi-axial pivoting of the anchor member 18 relative to the pivoting head 10. In other embodiments, the enlarged head 32 has other shapes to allow motion in fewer directions. For instance, a disc-shaped enlarged head 32 may provide motion within a desired plane. The enlarged head 32 may also include a driving feature 42 that allows a surgeon to attach the anchor member 18 to a vertebra V. In the embodiment shown, a hex recess driving feature 42 is shown. Other types of driving features 42 may be appropriate, including for example, slotted, star, Torx, and cross-shaped features. The driving feature 42 may be accessed through the aperture 26 shown in FIGS. 2A, 2B, and 3.

FIG. 5 shows a perspective view of a wear member 30 according to one embodiment. As depicted, the wear member 30 is cylindrically shaped and includes an outer surface 44 and an inner surface 46 extending between a top surface 50 and a bottom surface 52. Generally, the inner surface 46 is constructed to match the shape of the enlarged head 32 of the threaded anchor member 18. The outer surface 44 may be configured as desired to fit within the base portion 34 of the pivoting head 10 as shown in FIG. 3. In one embodiment, the outer surface 44 is substantially cylindrical.

The exemplary wear member 30 also includes a gap 48. The gap 48 in the present embodiment may be used to spread open the wear member 30 by an amount sufficient to slip the wear member 30 over the enlarged head 32 of the anchor member 18. The wear member 30 is shown installed on the enlarged head 32 in FIG. 6. FIG. 6 also shows relevant dimensions of the wear member 30 and the enlarged head 32. Dimension L represents a width of the enlarged head 32 at its widest point. Dimensions M and N respectively represent an interior width at the top 50 and bottom 52 of the wear member 30. Notably, dimension L is larger than both M and N. Thus, the gap 48 allows the enlarged head 32 to fit within the wear member 30 as shown in FIG. 6.

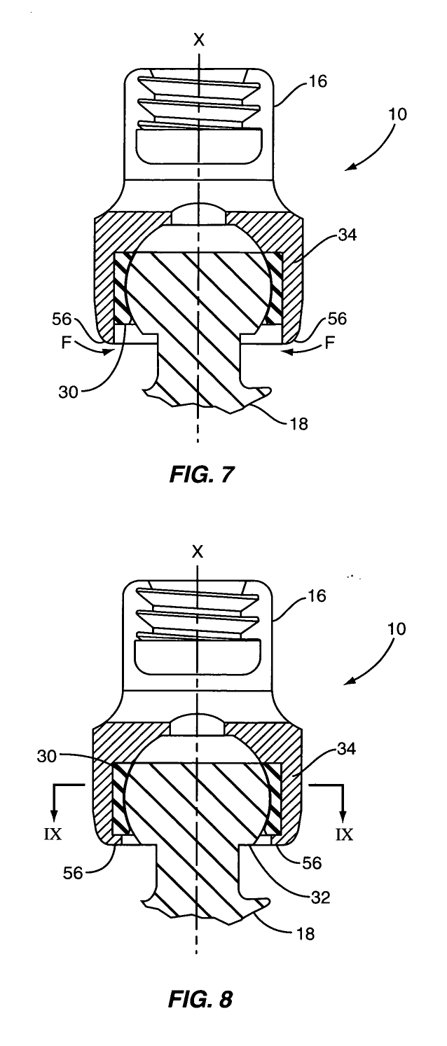

FIG. 7 shows the assembled wear member 30 and anchor member 18 inserted into the base portion 34 of the pivoting head 10. The anchor member 18 and wear member 30 are retained within the base portion 34 by deforming the lower lip 56 in the direction of the arrow labeled F. The deforming step may be performed using a variety of techniques, including but not limited to mechanical pressing, swaging, and orbital forming. Orbital forming (or orbital forging) is a cold metal forming process during which the workpiece (the base portion 34 in this case) is transformed between upper and lower dies. The process features one or the other of these dies orbiting relative to the other with a compression force applied therebetween. Due to this orbiting motion over the workpiece, the resultant localized forces can achieve a high degree of deformation at a relatively low compression force level. The fully assembled pivoting head 10 is illustrated in FIG. 8. In this figure, the lower lip 56 of the base portion 34 is formed to constrain the wear member 30 and the anchor member 18.

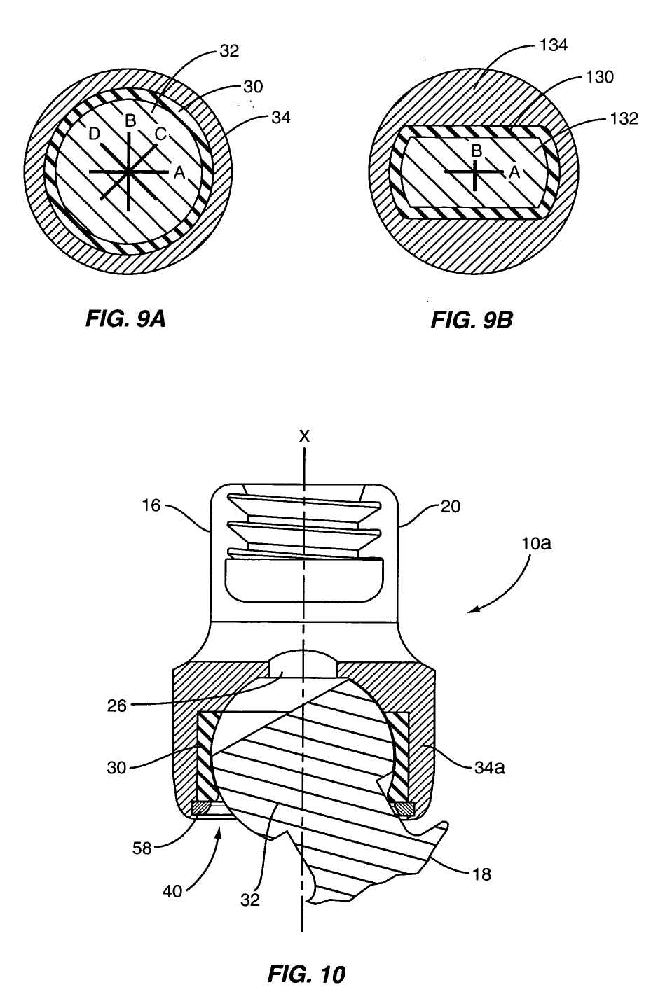

FIGS. 9A and 9B show section views according to the section line IX-IX shown in FIG. 8. FIG. 9A shows one embodiment where the enlarged head 32 and wear member 30 are substantially spherical as previously described. With this configuration, the pivoting head 10 may pivot about a plurality of axes, including axes A, B, C, and D as shown in FIG. 9A. FIG. 9B shows an alternative embodiment where the enlarged head 132 and wear member 130 are substantially disc-shaped. As disclosed above, this configuration may allow pivoting motion about axis B, but not other axes, including axis A.

FIG. 10 shows an alternative embodiment of the pivoting head 10a. The section view shown in FIG. 10 is similar to FIG. 8 and shows an alternative technique for retaining the wear member 30 and anchor member 18 within the base portion 34a. In this embodiment, a snap ring 58 is inserted into the bottom of the base portion 34a beneath the wear member 30. The snap ring 58 may effectively retain the wear member 30 and anchor member 18 within the pivoting head 10a.

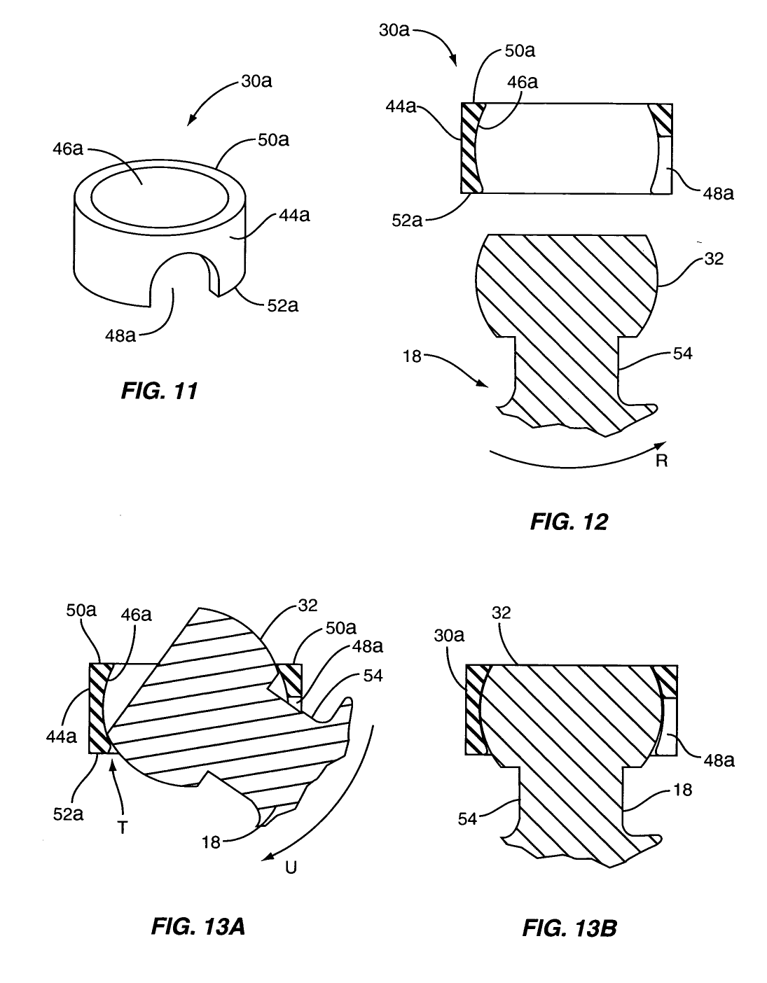

FIG. 11 shows an alternative configuration of the wear member 30a. The outer and inner surfaces 44a, 46a may be as described above. The wear member 30a also includes a gap 48a as with the previous embodiment shown in FIG. 5. However, gap 48a does not extend from the bottom surface 52a to the top surface 50a. In this embodiment, the top surface 50a of the wear member 30a is substantially continuous. The gap 48a is illustrated as an arc, though other shapes may be used. The gap 48a is sized to be wider than at least a top portion of the anchor member 18, just beneath the enlarged head 32, so that the anchor member 18 may be installed into the wear member 30a as shown in FIGS. 12, 13A, and 13B.

FIG. 12 shows a side cross-section view of the exemplary anchor member 18 and wear member 30a. In FIG. 12, the anchor member 18 and the wear member 30a are unassembled. To insert the anchor member 18 into the wear member 30a, the anchor member 18 is rotated (relative to the wear member 30a) in the direction of the arrow labeled R. Then, as shown in FIG. 13A, the enlarged head 32 of the rotated anchor member 18 is inserted into the wear member 30a. Also, with the anchor member 18 rotated as shown, a stem portion 54 of the anchor member 18 just beneath the enlarged head 32 is inserted into the gap 48a. The enlarged head 32 is inserted past the bottom surface 52a at point T. Once inserted in this manner, the anchor member 18 can be rotated back in the direction of the arrow labeled U and towards the orientation shown in FIG. 13B.

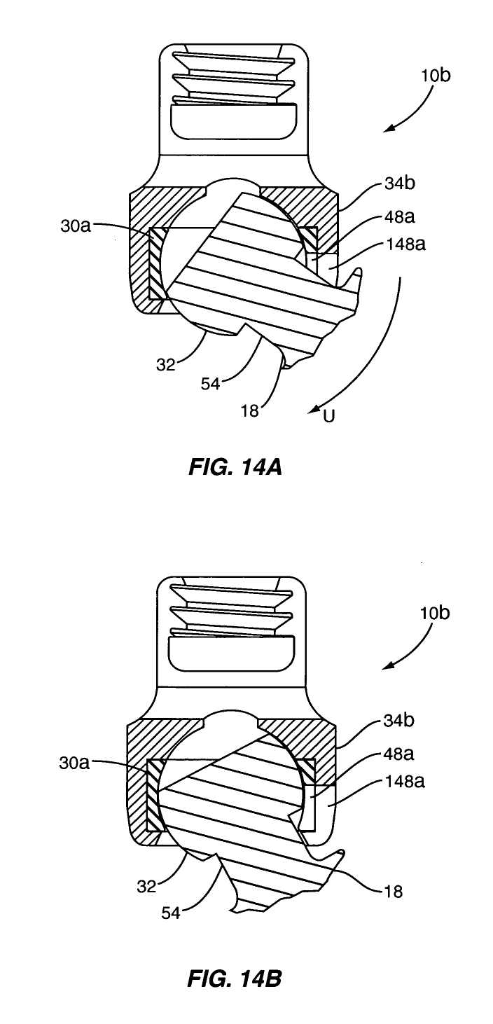

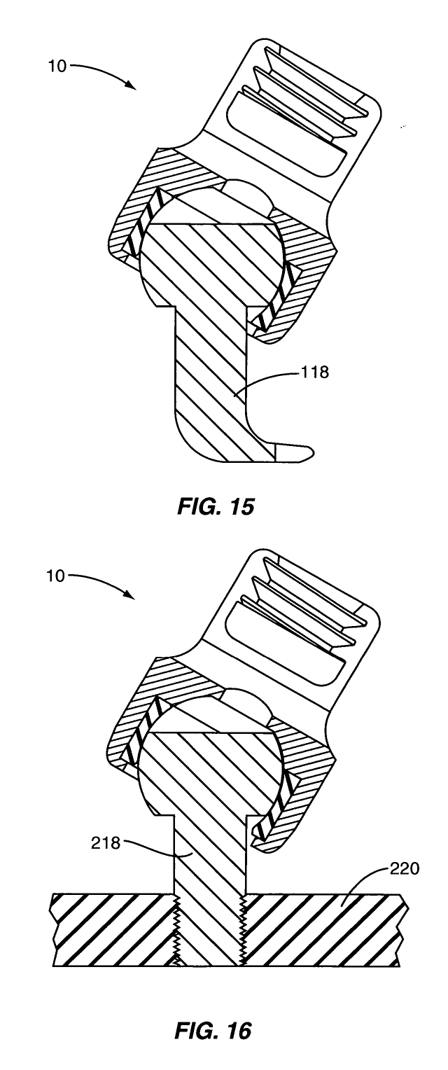

FIGS. 14A and 14B show an alternative embodiment of the pivoting head 10b where the anchor member 18 is inserted into the base portion 34b and wear member 30a in a manner similar to that depicted in FIGS. 13A and 13B. That is, to insert the anchor member 18 into the base portion 34b, the anchor member 18 is rotated approximately to the position shown in FIG. 14A. Then, the enlarged head 32 of the rotated anchor member 18 is inserted into the wear member 30a. At the same time, the stem portion 54 is inserted into the gap 48a and a gap 148a in the base portion 34b. Once inserted in this manner, the anchor member 18 can be rotated back in the direction of the arrow labeled U and towards the orientation shown in FIG. 14B.

Embodiments described above have contemplated an anchor member 18 that comprises threads for insertion into a vertebral member V. Certainly, the pivoting head 10 may be incorporated on other types of bone screws. For example, different types of screws may be used to attach longitudinal members 15 to the sacrum S or to other parts of a vertebral member V. These include, for example, anterior and lateral portions of a vertebral body. In other embodiments, such as those shown in FIGS. 15 and 16, the pivoting head 10 may be implemented on other types of anchoring members. For example, FIG. 15 shows a pivoting head 10 incorporated onto a hook-type anchor member 118. In another embodiment shown in FIG. 16, the pivoting head 10 is incorporated onto another type of threaded anchor member 218 that is inserted into a plate 220 instead of a bony member.

Spatially relative terms such as “under”, “below”, “lower”, “over”, “upper”, and the like, are used for ease of description to explain the positioning of one element relative to a second element. These terms are intended to encompass different orientations of the device in addition to different orientations than those depicted in the figures. Further, terms such as “first”, “second”, and the like, are also used to describe various elements, regions, sections, etc and are also not intended to be limiting. Like terms refer to like elements throughout the description.

As used herein, the terms “having”, “containing”, “including”, “comprising” and the like are open ended terms that indicate the presence of stated elements or features, but do not preclude additional elements or features. The articles “a”, “an” and “the” are intended to include the plural as well as the singular, unless the context clearly indicates otherwise.

The present invention may be carried out in other specific ways than those herein set forth without departing from the scope and essential characteristics of the invention. For example, embodiments described above have contemplated a pivoting head 10 having a substantially U-shaped recess in which to hold a longitudinal member 15. Certainly other types of configurations may incorporate the articulation mechanism 40 described herein. For example, alternative embodiments of the pivoting head may have circular apertures, C-shaped clamps, and multi-piece clamps as are known to secure a longitudinal member. The present embodiments are, therefore, to be considered in all respects as illustrative and not restrictive, and all changes coming within the meaning and equivalency range of the appended claims are intended to be embraced therein.

What is claimed is:

1. A connector to connect a vertebral member to a longitudinal member, the connector comprising:
an anchor comprising a shaft and an enlarged head;
a body attached to the anchor and comprising a receiver and a cavity that are aligned along a common axis, the receiver comprising a channel sized to receive the longitudinal member; and
a fastener configured to mate with the receiver to maintain the longitudinal member in the channel, a force applied by the fastener to maintain the longitudinal rod within the channel being isolated from the anchor; and
the cavity positioned on an opposite side of the body from the receiver and the cavity comprising a narrow opening that extends into an enlarged receiving area, the receiving area being isolated from the channel and sized to pivotally accommodate the head of the anchor with the narrow opening sized to retain the head within the receiving area.

2. The connector of claim 1, wherein the anchor is movably positioned within the body to pivot about the common axis.

3. The connector of claim 1, wherein the body further comprises an intermediate section positioned between the channel and the receiving area, the intermediate section and the body being constructed from a single member.

4. The connector of claim 3, wherein the intermediate section has a thickness to space apart the channel and the receiving area.

5. The connector of claim 1, wherein the receiving area further comprises a wear member that contacts the head of the anchor, the wear member being constructed of a different material from the body.

6. The connector of claim 5, wherein the wear member has an outer surface that contacts the body and an inner surface that contacts the head of the anchor.

7. The connector of claim 5, wherein the wear member has an outer surface that is constructed of a wear resistant coating.

8. The connector of claim 1, wherein a top section of the receiving area has a rounded configuration to conform with the head of the anchor.

9. The connector of claim 1, wherein the head of the anchor is constructed with a wear resistant coating.

10. A connector to connect a vertebral member to a longitudinal member, the connector comprising:
an anchor comprising a shaft and an enlarged head;
a body attached to the anchor and comprising a channel and a cavity aligned along a common axis, the channel sized to receive the longitudinal member;
a fastener configured to maintain the longitudinal member in the channel, a force applied by the fastener to maintain the longitudinal member within the channel being isolated from the anchor; and
a wear member positioned within the cavity and constructed from a material different from the body, the wear member forming a receiving area sized to pivotally accommodate the head of the anchor;
the cavity comprising a narrow opening to retain the head within the receiving area and the receiving area positioned for the anchor to pivot when the fastener maintains the longitudinal member in the channel.

11. The connector of claim 10, wherein the head contacts the wear member when the anchor pivots within the body.

12. The connector of claim 10, wherein the wear member is a coating applied to an inner surface of the cavity.

13. The connector of claim 10, wherein the wear member comprises a first surface that contacts an inner surface of the cavity, and a second surface that contacts the head of the anchor.

14. The connector of claim 10, wherein an adhesive attaches the wear member to an inner surface of the cavity.

15. The connector of claim 10, wherein the wear member has a width that is greater than the narrow opening to maintain the wear member within the cavity.

16. The connector of claim 10, wherein the anchor is movably positioned within the wear member to pivot about the common axis.

17. The connector of claim 10, wherein the body further comprises an intermediate section positioned between the channel and the cavity, the intermediate section and the body being constructed from a single member.

18. The connector of claim 10, wherein a top section of the cavity comprises a stop to prevent the wear member from pivoting within the cavity during movement of the anchor.

19. The connector of claim 10, wherein the head of the anchor is constructed with a wear resistant coating.

20. The connector of claim 10, wherein the wear member is constructed with a wear resistant coating.

21. A connector to connect a vertebral member to a longitudinal member, the connector comprising:
an anchor comprising a shaft and an enlarged head;
a body attached to the anchor and being constructed from a single member having a receiver, a cavity, and an intermediate section, the receiver comprising a channel sized to receive the longitudinal member; and
a fastener configured to mate with the receiver to maintain the longitudinal member in the channel;
the cavity and channel being aligned on a common axis and positioned on opposite sides of the intermediate section, the cavity comprising a narrow opening that extends into an enlarged receiving area, the receiving area being isolated from the channel and sized to accommodate the head of the anchor, and the narrow opening being sized to retain the head within the receiving area;
the receiving area being isolated from the channel and sized to allow the anchor to freely pivot when the fastener mates with the receiver.

22. The connector of claim 21, wherein the intermediate section is substantially perpendicular to the common axis.

23. The connector of claim 21, further comprising a wear member positioned within the receiving area to contact the head of the anchor, the wear member constructed of a different material than the body.

24. The connector of claim 23, wherein the different material comprises a wear resistant coating.

25. The connector of claim 21, wherein the anchor is movably positioned within the body to pivot about the common axis.

26. The connector of claim 21, wherein a top section of the receiving area has a rounded configuration to conform with the head of the anchor.

27. The connector of claim 21, wherein the head of the anchor is constructed with a wear resistant coating.
